(19) United States
(12) Patent Application Publication
(10) Pub. No.: US 2011/0313160 A1
(43) Pub. Date: Dec. 22, 2011

(54) PREPARATION OF 5-ETHYL-2--PYRIMIDINE

(75) Inventors: Xin Chen, Hayward, CA (US); Jingyuan Ma, Hayward, CA (US); Jiangao Song, Hayward, CA (US); Imad Nashashibi, Hayward, CA (US)

(73) Assignee: Metabolex, Inc.

(21) Appl. No.: 13/152,752

(22) Filed: Jun. 3, 2011

Related U.S. Application Data
(60) Provisional application No. 61/351,803, filed on Jun. 4, 2010.

(51) Int. Cl. C07D 417/14; C07D 401/04

(52) U.S. Cl. 544/331

(57) ABSTRACT

Processes and intermediates for the synthesis of 5-ethyl-2-{4-[4-(4-tetrazol-1-yl-phenoxymethyl)-thiazol-2-yl]-piperidin-1-yl}-pyrimidine are provided.

### CROSS-REFERENCE TO RELATED APPLICATIONS

This application claims benefit under 35 U.S.C. §119(e) to application Ser. No. 61/351,803, filed Jun. 4, 2010, the content of which is incorporated herein by reference in its entirety.

### TECHNICAL FIELD

The present invention relates to the field of pharmaceutical chemistry.

### BACKGROUND

Pyrimidine compounds useful for treatment of diabetes and other metabolic disorders are disclosed in U.S. Pat. No. 7,638,541. One such compound is 5-ethyl-2-{4-[4-(4-tetrazol-1-yl-phenoxymethyl)-thiazol-2-yl]-piperidin-1-yl}-pyrimidine and salts thereof.

### SUMMARY OF THE INVENTION

The present invention provides processes and intermediates for the improved synthesis of 5-ethyl-2-{4-[4-(4-tetrazol-1-yl-phenoxymethyl)-thiazol-2-yl]-piperidin-1-yl}-pyrimidine and pharmaceutically acceptable salts thereof.

### DETAILED DESCRIPTION OF THE INVENTION

In one embodiment, provided is a method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof

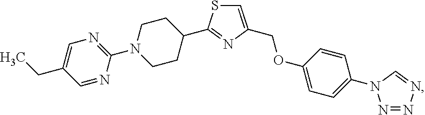

the method comprising contacting in dimethylformamide in presence of base a compound of Formula (IX) with a compound of Formula (X) wherein L is a leaving group such as F, Cl, Br, I, OS(O)₂CF₃, OS(O)₂CH₃ and OS(O)CF₃.

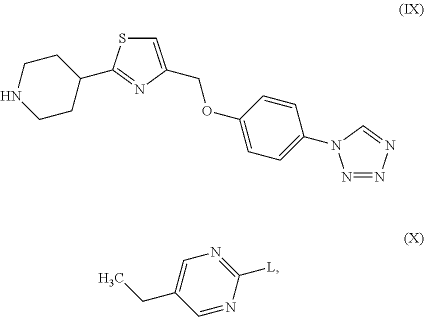

and optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

In some aspects, the compound of Formula (IX) and (X) are contacted at a temperature of 60° C. to 100° C. In other aspects, the temperature is 70° C. to 90° C., 79° C. to 81° C., or 80° C.

In some aspects, the base is NaOH, Na₂CO₃, NaHCO₃, KHCO₃, K₂CO₃, Cs₂CO₃, Et₃N (triethylamine) and i-Pr₂NEt.

In some embodiments, the compound of Formula (IX) is prepared by contacting a compound of Formula (VIII) with acid

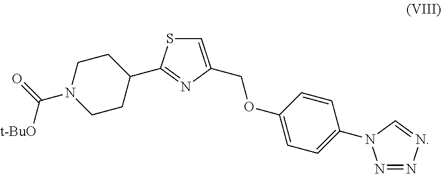

In some embodiments, the compound of Formula (VIII) is prepared by contacting a compound of Formula (VI) with a compound of Formula (VII)

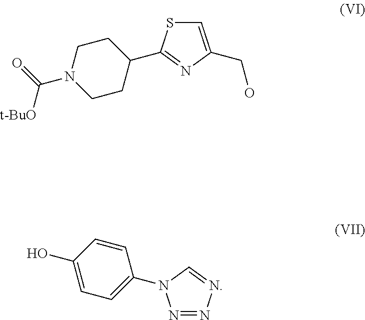

In some aspects, the compound of the compounds of Formula (VI) and Formula (VII) are contacted in a polar organic solvent selected from dimethyl formamide (DMF) and acetonitrile (MeCN) and in presence of base. In some aspects, the base is selected from the group consisting of NaOH, Na₂CO₃, K₂CO₃, Cs₂CO₃ and NaH.

In some aspects, the compound of the solvent is MeCN. In other aspects, the solvent is DMF.

In some aspects, the base is Cs₂CO₃. In still other aspects the base is K₂CO₃.

In some embodiments, the compound of Formula (VI) is prepared by contacting a compound of Formula (IV) with a compound of Formula (V)

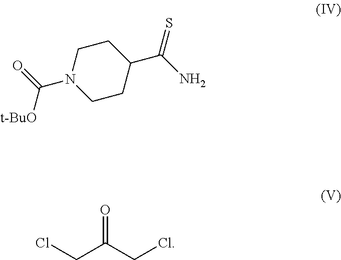

In some aspects, the compounds of Formula (IV) and Formula (V) are refluxed in a polar organic solvent in presence of base. In some such aspects, the base is selected from the group consisting of Na₂CO₃, K₂CO₃, Cs₂CO and MgCO₃.

In some embodiments, the compound of Formula (VII) is prepared by contacting 4-aminophenol with sodium azide and trimethylorthoformate.

In some embodiments, the compound of Formula (IV) is prepared by contacting a compound of Formula (II) with a compound of Formula (III)

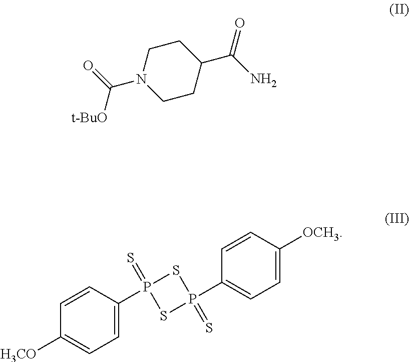

In some embodiments, the compound of Formula (II) is prepared by contacting a compound of Formula (I) with di-tert-butyl dicarbonate (Boc₂O).

In one embodiment provided is a method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof

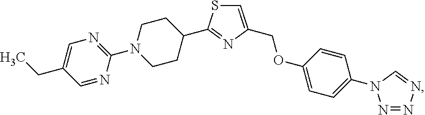

the method comprising:

(a) contacting a compound of Formula (I) with di-tert-butyl dicarbonate (Boc₂O) to form a compound of Formula (ID

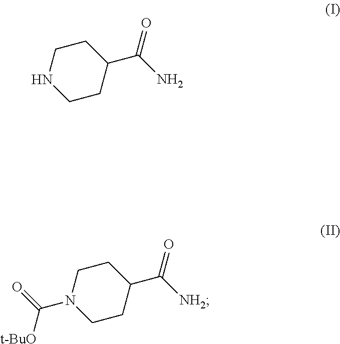

(b) contacting the compound of Formula (II) with a compound of Formula (III) to form a compound of Formula (IV)

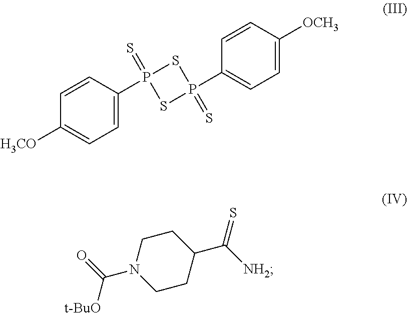

(c) contacting the compound of Formula (IV) with a compound of Formula (V) to form a compound of Formula (VI)

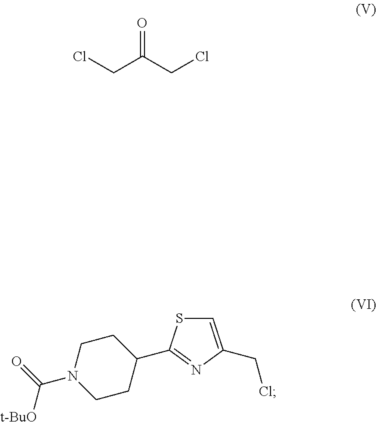

(d) contacting the compound of Formula (VI) with a compound of Formula (VII) to form a compound of Formula (VIII)

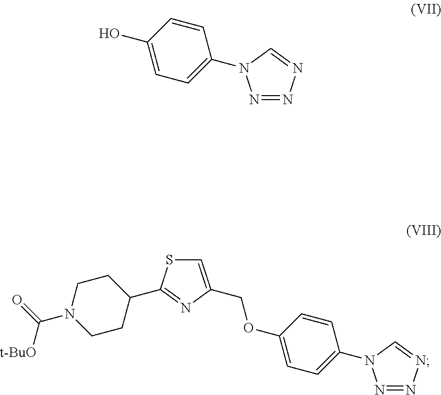

(e) contacting the compound of Formula (VIII) with acid to form a compound of Formula

(IX)

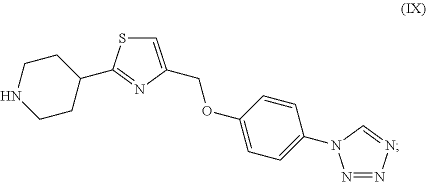

(f) contacting in dimethylformamide in presence of base the compound of Formula (IX) with a compound of Formula (X) wherein L is a leaving group such as F, Cl, Br, I, OS(O)₂CF₃, OS(O)₂CH₃ and OS(O)CF₃

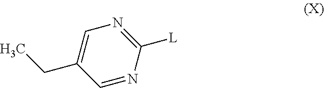

to form 5-ethyl-2-{4-[4-(4-tetrazol-1-yl-phenoxymethyl)-thiazol-2-yl]-piperidin-1-yl}-pyrimidine; and

(g) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

In one embodiment provided is method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof

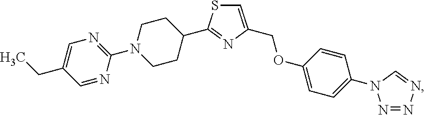

comprising contacting a compound of Formula (XXIV) with a compound of Formula (VII) in presence of base, such as NaOH, Na₂CO₃, K₂CO₃, Cs₂CO₃ and NaH

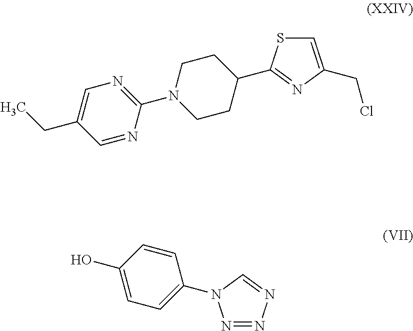

and optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

In one embodiment provided is a method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof.

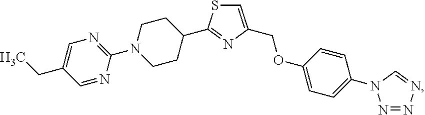

the method comprising:

(a) contacting a compound of Formula (I) with a compound of Formula (XXI) wherein T is a leaving group such as F, Cl, Br, I, OS(O)₂CF₃, OS(O)₂CH₃ and OS(O)CF₃ to form a compound of Formula (XXII)

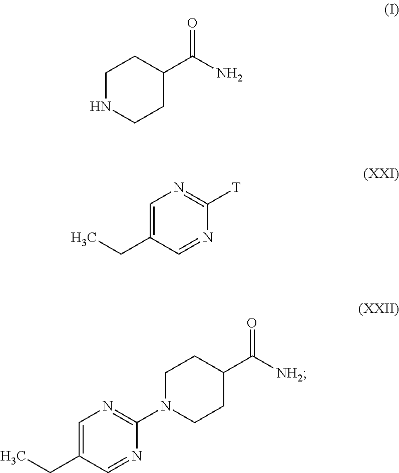

(b) contacting the compound of Formula (XXII) with a compound of Formula (III) to form a compound of Formula (XXIII)

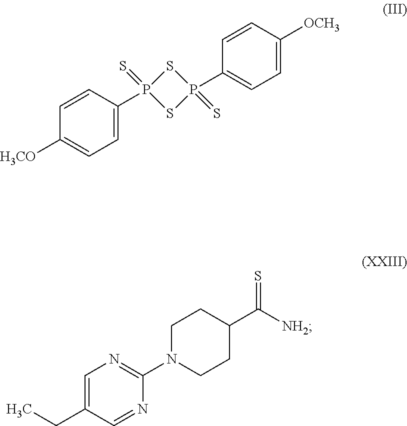

(c) contacting the compound of Formula (XXIII) with a compound of Formula (V) to form a compound of Formula (XXIV)

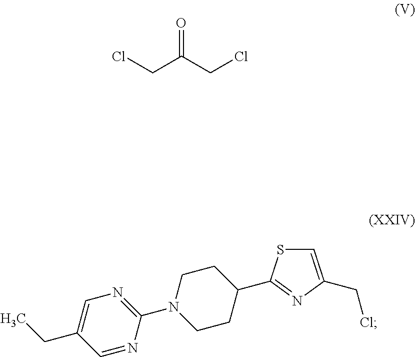

(d) contacting the compound of Formula (XXIV) with a compound of Formula (VII)

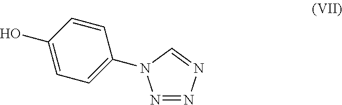

to form 5-ethyl-2-{4-[4-(4-tetrazol-1-yl-phenoxymethyl)-thiazol-2-yl]-piperidin-1-yl}-pyrimidine; and

(e) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

In one embodiment provided is a method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof

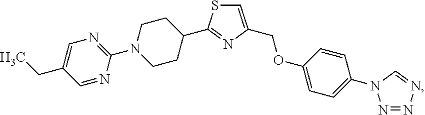

the method comprising:

(a) contacting a compound of Formula (IV) with acid to form a compound of Formula (XI)

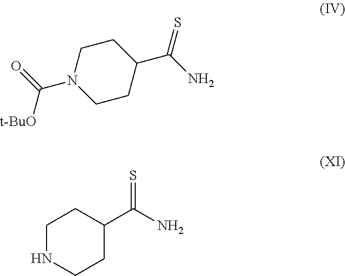

(b) contacting a compound of Formula (XXI) wherein T is a leaving group such as F, Cl, Br, I, OS(O)₂CF₃, OS(O)₂CH₃ and OS(O)CF₃, to form a compound of Formula (XXIII)

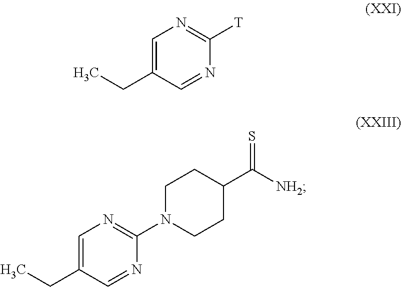

(c) contacting the compound of Formula (XXIII) with a compound of Formula (V) to form a compound of Formula (XXIV)

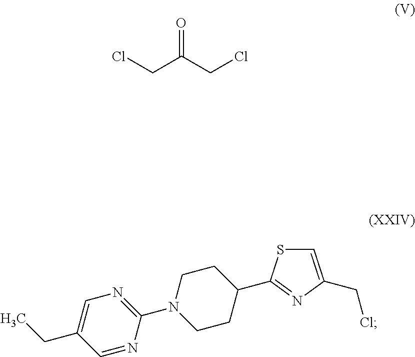

(d) contacting the compound of Formula (XXIV) with a compound of Formula (VII)

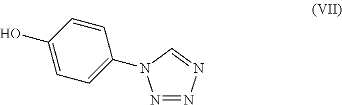

to form 5-ethyl-2-{4-[4-(4-tetrazol-1-yl-phenoxymethyl)-thiazol-2-yl]-piperidin-1-yl}-pyrimidine; and

(e) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

In one embodiment provided is a method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof

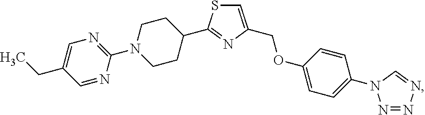

the method comprising:

(a) contacting the compound of Formula (XOH) with a compound of Formula (XXIV) to form a compound of Formula (XXV)

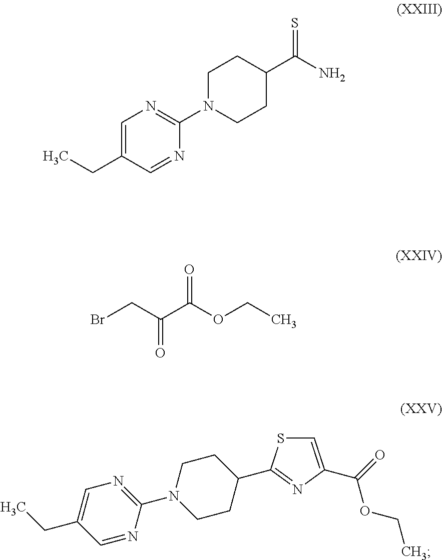

(b) contacting the compound of Formula (XXV) with a reducing agent, for example lithium aluminum hydride (LiAlH₄), lithium borohydride (LiBH₄), or diisobutyl aluminum hydride (DiBal) to form a compound of Formula (XXVI)

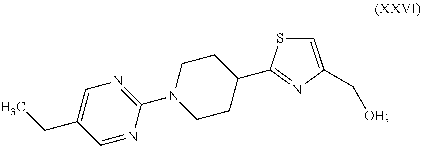

(c) contacting the compound of Formula (XXVI) with a compound of Formula (VII)

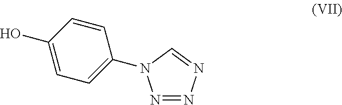

under Mitsunobu coupling conditions to form 5-ethyl-2-{4-[4-(4-tetrazol-1-yl-phenoxymethyl)-thiazol-2-yl]-piperidin-1-yl}-pyrimidine; and

(d) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

In one embodiment provided is a method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof

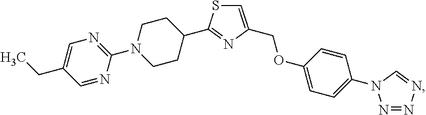

the method comprising:

(a) contacting the compound of Formula (XOH) with a compound of Formula (XXIV) to form a compound of Formula (XXV)

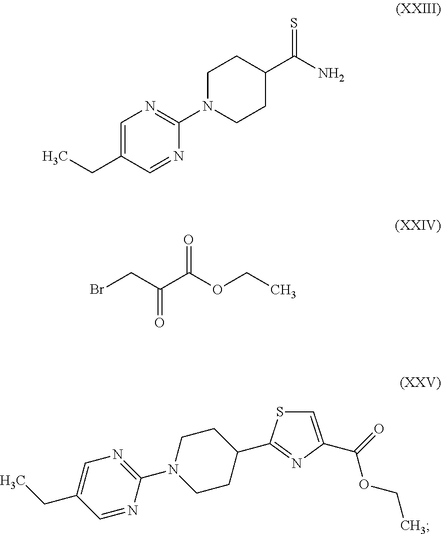

(b) contacting the compound of Formula (XXV) with a reducing agent to form a compound of Formula (XXVI)

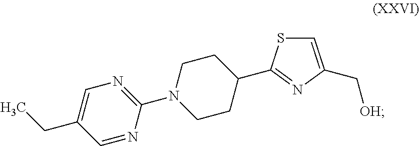

(c) converting the compound of Formula (XXVI) to a compound of Formula (XXVII) wherein Q is a leaving group such as Cl, Br, I, OS(O)₂CF₃, OS(O)₂CH₃ and OS(O)CF₃.

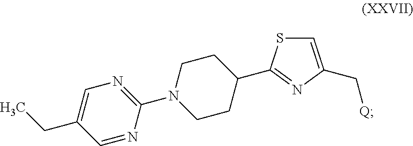

(d) contacting the compound of Formula (XXVII) with a compound of Formula (VII)

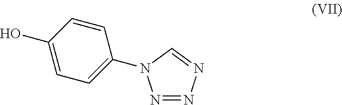

to form 5-ethyl-2-{4-[4-(4-tetrazol-1-yl-phenoxymethyl)-thiazol-2-yl]-piperidin-1-yl}-pyrimidine; and

(e) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

In one embodiment provided is a method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof

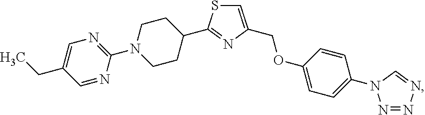

comprising contacting a compound of Formula (XXVII) wherein Q is a leaving group such as Cl, Br, I, OS(O)₂CF₃, OS(O)₂CH₃ and OS(O)CF₃ with a compound of Formula (VII) in presence of base, for example NaOH, Na₂CO₃, K₂CO₃, Cs₂CO₃ and NaH.

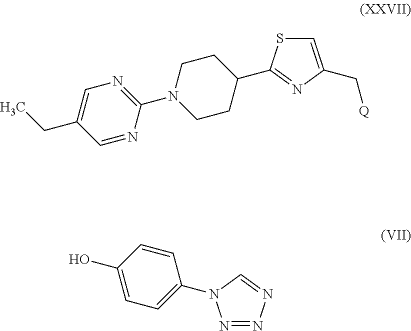

and optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

In some aspects, the processes disclosed herein provide a pharmaceutically acceptable salt of 5-ethyl-2-{4-[4-(4-tetrazol-1-yl-phenoxymethyl)-thiazol-2-yl]-piperidin-1-yl}-pyrimidine. In some such aspects the salt is a HCl salt.

In some aspects provided is an intermediate compound for use in the preparation of 5-ethyl-2-{4-[4-(4-tetrazol-1-yl-phenoxymethyl)-thiazol-2-yl]-piperidin-1-yl}-pyrimidine selected from the group consisting of

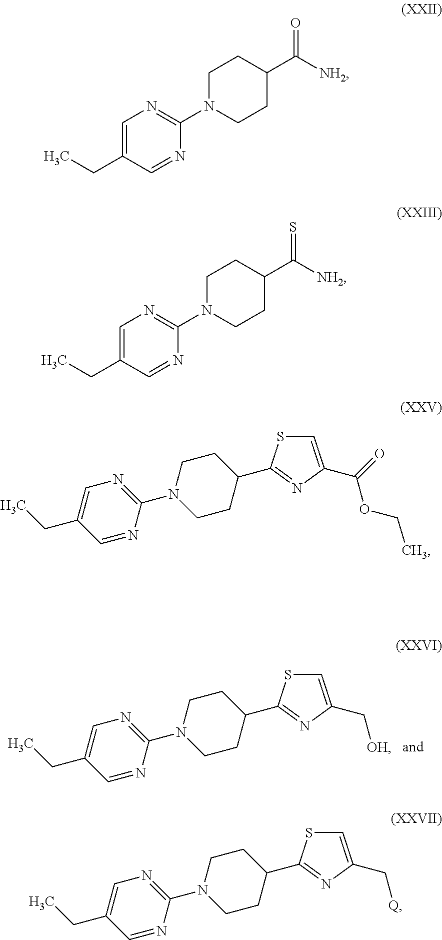

wherein Q is a leaving group such as Cl, Br, I, OS(O)₂CF₃, OS(O)₂CH₃ and OS(O)CF₃.

In other embodiments, provided is 5-ethyl-2-{4-[4-(4-tetrazol-1-yl-phenoxymethyl)-thiazol-2-yl]-piperidin-1-yl}-pyrimidine having carbon 14 isotope labeling about the carbon atoms in the phenyl ring. The labeled compound can be prepared according to the following scheme from commercially available 14C(U)]-4-aminophenol hydrochloride (Archemi 1-800-331-6661, ARC-545):

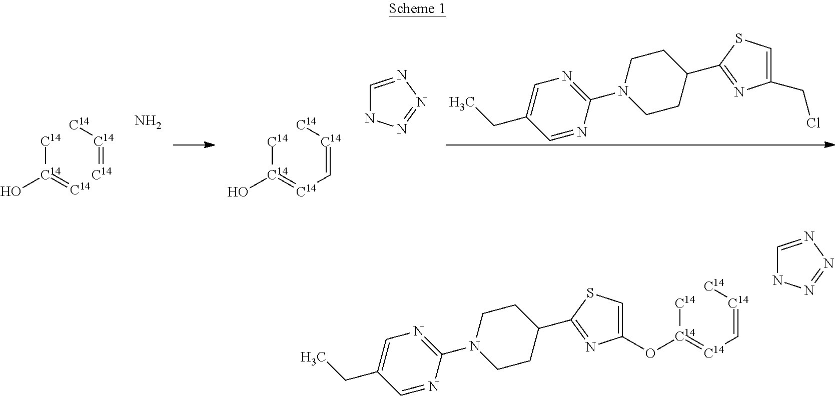

The present invention will be described in further detail by the following examples. It is to be understood, however, that these examples are given for illustrative purpose only and are not construed to limit the scope of the present invention.

### Example 1

### 4-Carbamoyl-piperidine-1-carboxylic acid tert-butyl ester

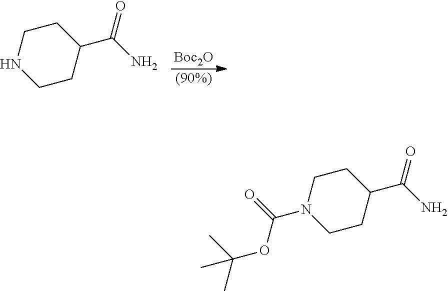

To a suspension of iosnipecotamide (255 g, 1.99 mol) and 4-dimethylamino-pyridine (204 mg, 1.82 mol) in methylene chloride (1500 mL) in a 5-lite of three-neck flask was added a solution of di-tert-butyl dicarbonate (502 g, 2.30 mol, 1.15 eq.) in methylene chloride (500 mL) dropwise at room temperature with mechanic stirring. A clear solution was reached at the end of the adding. After stirring at room temperature for two more hours, the solution was washed with phosphoric acid water solution (2.5 v/v %, 500 mL), water (500 mL), half saturated sodium bicarbonate water solution (500 mL), and 10% of brine (500 mL). The organic phase was dried over anhydrous sodium sulfate. During the course of removing of the methylene chloride, ethyl acetate (100 ml) and heptane (200 mL) was added. After removing the methylene chloride, the white solid formed was filtrated, washed with hexane, and dried to give 414 g (95%) of product.

TLC: dichloromethane-methanol 90:10, Rf (product)=0.28; Rf (starting material)=base line, iodine positive.

### Example 2

### 4-Thiocarbamoyl-piperidine-1-carboxylic acid tert-butyl ester

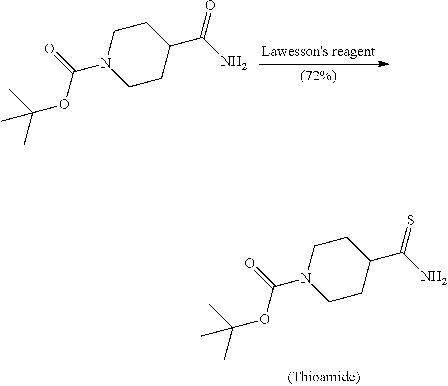

To a suspension of 4-Carbamoyl-piperidine-1-carboxylic acid tert-butyl ester (288 g, 1.26 mol) in dimethoxyethane (2000 mL) and methylene chloride (800 mL) in a 5-lite of three-neck flask was added Lawesson's Reagent (255 g, 0.63 mol). The mixture was stirred at room temperature for 80 min. TLC check there was no starting material left. The solvents were removed under vacuum. The residue was dissolved in ethyl acetate (1500 mL), and washed with half saturated potassium carbonate water solution (500 mL each, two times), 50% of brine (500 mL). The organic phase was dried over anhydrous sodium sulfate and concentrated to dry. The obtained solid was dissolved in ethyl acetate (1000 mL) and filtered at hot to remove insoluble white stuff. To the solution was added heptane (300 mL). After removing most of ethyl acetate, the solid formed was filtrated, washed with hexane-ether (1:1), and dried to give 252 g (82%) of product.

TLC: dichloromethane-methanol 90:10, Rf (product)=0.37, UV and iodine positive; Rf (starting material)=0.28, iodine positive.

### Example 3a

### 4-Tetrazol-1-ly-phenol

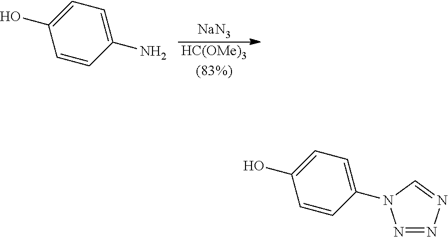

To a 2-liter one-neck flask under air, immersed in an oil bath and fitted with a refluxing condenser, was added 4-aminophenol (50 g, 0.459 mol), acetic acid (500 mL), sodium azide (41.7 g, 0.642 mol), and trimethyl orthoformate (70 mL, 68 g, 0.642 mol). The mixture was stirred at 60° C. (oil bath) for one hour and then refluxed (oil bath, 100° C.) for 3 hours. A clear solution was formed during the refluxing. The temperature of solution was lowered to 80° C. (oil bath) and water (300 mL) was added slowly. The temperature of the solution was cooled down to room temperature. The solid formed over night was filtered and dried to give 61.7 g (83%) of product as first crop.

TLC: hexane-ethyl acetate 50:50, Rf (product)=0.28; Rf (starting material)=0.23, UV and iodine positive.

¹HNMR (400 MHz, D₃COD), δ 9.58 (s, 1H), 7.61 (d, J=9.0 Hz, 2H), 6.97 (d, J=9.0 Hz, 2H) ppm.

Modified procedure: The reactions were carried out at 1.5 times of the above-mentioned scale. A 2-liter flask under air was charged with acidic acid followed by 4-aminophenol, sodium azide, and trimethyl orthoformate with stirring at room temperature. The flask was fitted with a bump trap and was heated to 100° C. (oil bath) during the course of 1 to 1.5 hours. Solid started to precipitate and the temperature of mixture was lowered to 80° C. Water was added and the mixture was cooled down to room temperature. The mixture was filtered and the solid was washed with water and dried to give the desired product (>88% yield).

¹HNMR (400 MHz, D₃COD), δ 9.58 (s, 1H), 7.61 (d, J=9.0 Hz, 2H), 6.97 (d, J=9.0 Hz, 2H) ppm.

### Example 3b

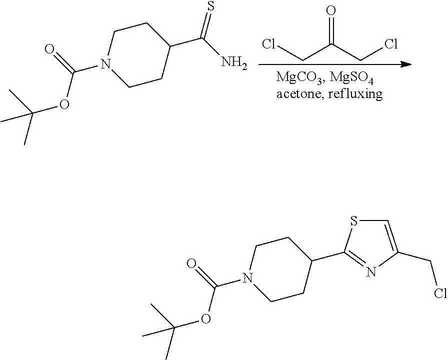

To a 500 mL flask under air, immersed in an oil bath and a condenser, was added 4-thiocarbamoyl-piperidine-1-carboxylic acid tert-butyl ester (29 g, 120 mmol), acetone (300 mL) MgSO₄ (21.6 g, 180 mmol) and MgCO₃ (10 g, 120 mmol), 1,3-dichloroacetone (19.8 g, 156 mmol). The resulting mixture was heated under reflux overnight, cooled and filtered through celite. The solvent was removed in vacuo and the residue was redissolved with EtOAc (500 mL). The resulting solution was washed successively with 5% NaHSO₃ (twice), saturated NaHCO₃ and brine. After drying (NaSO₄), the solvent was removed to afford 35 g of the title compound as light yellow oil. The oil became dark solid after standing at room temperature. The color could be removed by activated charcoal. The purity was improved from 92% to 96%.

¹H NMR (CDCl₃): δ 7.20 (1H, s), 4.67 (2H, s), 4.20 (2H, br), 3.16 (1H, m), 2.87 (2H, m), 2.09 (2H, m), 1.72 (2H, m), 1.47 (9H, s).

### Example 4

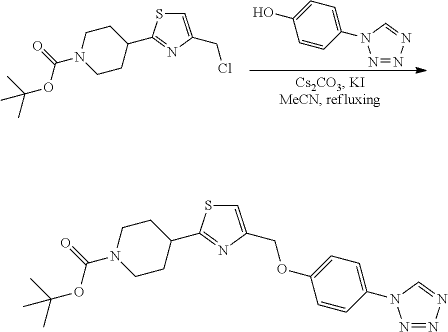

A mixture of 4-(4-chloromethyl-thiazol-2-yl)-piperidine-1-carboxylic acid tert-butyl ester (35 g, 0.11 mol), 4-tetrazol-1-yl-phenol (21.4 g, 0.132 mol), Cs₂CO₃ (43 g, 0.132 mol), KI (1.8 g, 11 mmol) in acetonitrile (400 mL) was heated under reflux overnight. After cooling, the solid was filtered through a pad of celite. The filtrate was concentrated in vacuo. The residue was dissolved in methylene chloride and washed with 5% aqueous NaOH (3 times), water and brine. After drying (NaSO₄), the solvent was removed. The resulting solid was dissolved in ethyl acetate. The resulting solution was heated with activated charcoal and filtrated through a pad of celite. The filtrate was concentrated and the residue was purified by recrystallization from EtOAc/Hexane to afford 37 g desired product.

¹H NMR (CDCl₃): δ 8.01 (1H, s), 7.61 (2H, d, J=8.8 Hz), 7.25 (1H, s), 7.15 (2H, d, J=8.8 Hz), 5.22 (2H, s), 4.2 (2H, br), 3.17 (1H, m), 2.87 (2H, m), 2.11 (2H, m), 1.73 (2H, m), 1.46 (9H, s).

### Example 5

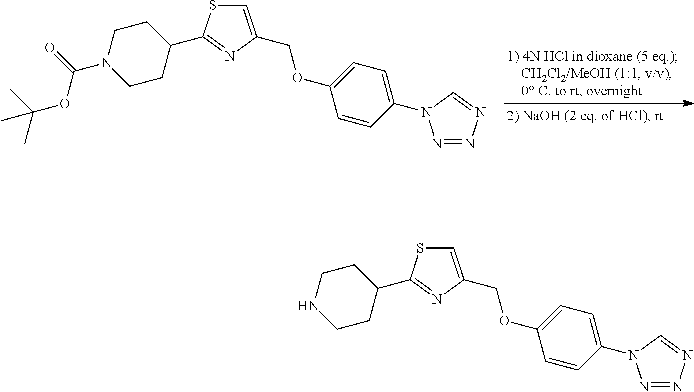

To a 3-L 3-neck flask under N₂ fitted with an addition funnel, was added 400 mL of anhydrous methylene chloride (J. T. Baker low water grade; the CH₂Cl₂ will facilitate the solubility of substrate) and 115.59 g of t-butyl carbamate substrate (0.26 mol) in one-portion. After stirring at rt for 2˜5 minutes, to the resulting almost clear solution was added 400 mL of methanol (J. T. Baker HPLC grade). The resulting clear brown solution was cooled to 0-4° C. (ice-water bath temperature) with stirring, and then 330 mL of 4N HCl in 1,4-dioxane (1.32 mol, 5 eq.) was added dropwise over 30 minutes. The ice-water bath was removed, and the resulting brown homogeneous solution was stirred at rt overnight (15 hours). At least 7 hours is needed to bring the reaction to completion. The reaction mixture was aliquoted and quenched into 2N NaOH, and then extracted w/EtOAc. ¹H NMR in DMSO-d₆. Diagnostic peaks: free-amine product δ 7.63 (s, JH); starting material (substrate) δ 7.66 (s, JH). Typically, the conversion was estimated via the integral of the italicized signals: 4 hrs, 80% conversion; 6 hrs, 95% conversion. The reaction solution was allowed to cool to 10° C. (ice-water bath temp), and then a solution of 15% (w/v) NaOH (705 mL; 2.64 mol, 2 eq. of HCl used) in ˜500 mL of water was added dropwise over 15 minutes. (Diluted 15% aq. NaOH was used to ensure no precipitation (inorganic salt) in the organic phase). Immediate phase break was observed when the stirring was stopped to give a brown aqueous layer on top and a pale yellow organic layer on the bottom. The organic layer was collected, and the remaining aqueous layer was extracted with CH₂Cl₂ (500 mL×2). The organic layers were combined, rinsed with 500 mL of water, and dried over anhy. Na₂SO₄. After most of solvents were removed in vacuo, precipitation began. To this pale yellow mixture was added 500 mL of heptane to give a pale yellow slurry. The resulting precipitate was collected on a filter funnel, and the mother liquor was stripped down. The combined solids were rinsed with heptane (200 mL). After air-drying overnight, 84.1 g (94% yield) of free amine was obtained as a white or an off-white solid.

¹H NMR (DMSO-d₆): δ 9.98 (1H, s), 7.80 (2H, d, J=8.0 Hz), 7.63 (1H, s), 7.28 (2H, d, J=8.0 Hz), 5.20 (2H, s), 3.05 (1H, m), 2.97 (2H, m), 2.56 (2H, m), 1.93 (2H, m), 1.55 (2H, m) ppm.

Instead of using HCl, if the reaction was treated with 5 eq. TFA in CH₂Cl₂ at rt, ˜50% of an unknown by-product will be generated which can be seen by taking a ¹H NMR in DMSO-d₆: Diagnostic peaks δ 7.45 (1H, s), 6.61 (2H, d, J=8.8 Hz), 6.44 (2H, d, J=8.8 Hz), 4.89 (2H, s) ppm. The use of CH₂Cl₂/CH₃OH as co-solvents will eliminate the formation of impurities seen with other solvents. The use of 1,4-dioxane, 1,4-dioxane/methanol, or methylene chloride will produce a tiny amount of detectable impurity which can be seen by ¹H NMR in DMSO-d₆: Diagnostic peaks δ 6.82 (m), 6.56 (m), 4.99 (m) ppm. This impurity will be carried over to the final product in the next step, and cannot be removed by purification via recrystallization.

### Example 6

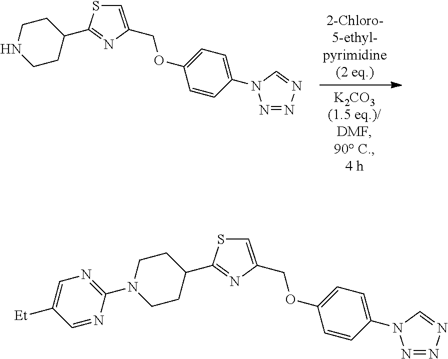

To a 3-L 3-neck flask under N₂ was added 105.7 g of crude free amine (0.31 mol), 88.0 g of 2-chloro-5-ethylpyrimidine (0.62 mol, 2 eq.) in one-portion, and then 800 mL of anhydrous DMF. After stirring at rt for 1˜2 minutes, to the resulting clear solution was added 64.0 g of anhy. K₂CO₃ (0.46 mol, 1.5 eq.) in one-portion. The flask was immersed in a pre-heated oil bath (90° C., oil-bath temperature), and the reaction mixture was stirred at 90° C. (oil-bath temperature) for 3.5 hours. The reaction mixture was aliquoted and quenched into water/brine, and then extracted w/EtOAc. ¹H NMR in DMSO-d₆. Diagnostic peaks: product δ 7.66 (s, 1H); free-amine (starting material) δ 7.63 (s, 1H); pyrimidine δ 8.67 (s, 2H), DMF δ 7.03 (s, 1H). Typically, the conversion was estimated via the integral of the italicized signals. Complete conversion was observed between 3 to 4 hours. Prolonged heating (>5 hours) resulted in the formation of the unidentified impurity.

The reaction mixture was transferred to a 5-L 3-neck flask, and allowed to cool with stirring to rt with ice-water bath. To the reaction mixture at rt under stirring vigorously (mechanical stirrer) and approximate 2000 mL of water was added slowly dropwise over 30 minutes to give an off-white slurry (precipitation began when ˜500 mL of water was added). After the addition was finished, the resulting slurry was stirred at rt for an additional 1015 minutes. The off-white precipitate was filtered and then rinsed with water (250 mL×2). After air-drying overnight, approximate 387 g of wet off-white solid was obtained, and redissolved in 1500 mL of EtOAc by heating at 55° C. (internal solution temperature) for ca. 10 minutes. The resulting pale-yellow solution was washed with water (250 mL×3) and water/brine (200 mL/100 mL), and dried over anhy. Na₂SO₄. After most of solvents were removed in vacuo, precipitation began and then gave an off-white slurry (˜500 mL of solvents left). The resulting white precipitate was collected on a filter funnel, and rinsed with EtOAc (300 mL×2). The mother liquor was kept to do another recrystallization later on, and the precipitate on the filter funnel was rinsed once more time with 300 mL of heptane. After air-drying, 91.11 g of product was obtained as a white solid. The mother liquor (without heptane) was stripped down in vacuo until a thick slurry was formed, and the resulting precipitate was filtered and rinsed twice with EtOAc (100 mL×2) and once with heptane (100 mL) to give another 16.84 of product as a white solid. Overall yield 78%.

¹H NMR (DMSO-d₆): δ 9.98 (1H, s), 8.24 (2H, s), 7.80 (2H, d, J=6.8 Hz), 7.66 (1H, s), 7.28 (2H, d, J=6.8 Hz), 5.20 (2H, s), 4.67 (2H, m), 3.32 (1H, m), 3.01 (2H, m), 2.43 (2H, q, J=7.2 Hz), 2.07 (2H, m), 1.59 (2H, m), 1.11 (3H, t, J=7.2 Hz) ppm. All the remaining mother liquors were combined, and concentrated in vacuo to give 15.07 g of an off-white solid which would be purified by one more time recrystallization with EtOAc or chromatography with 70% EtOAc/hexanes on silica gel.

This reaction was also tried at a small scale (0.6 mmol) at higher concentrations (0.6 M with 2 eq. of pyrimidine and 1.2 M with 1.3 eq. of pyrimidine).

Free amine (207 mg, 0.60 mmol) was treated at 90° C. with 178.3 mg of 2-chloro-5-ethylpyrimidine (2 eq.) and anhy. K₂CO₃ (1.5 eq.) in 1 mL of DMF (the final concentration of the free amine is ˜0.60 M). The reaction was complete in 2 hours. However, the reaction mixture was not homogenous at the end because of the precipitation of product.

Free amine (212 mg, 0.62 mmol) was treated at 90° C. with 114.2 mg of 2-chloro-5-ethylpyrimidine (1.3 eq.) and anhy. K₂CO₃ (1.5 eq.) in 0.5 mL of DMF (the final concentration of the free amine is ˜1.2 M). The reaction was achieved ˜85% conversion in 2 hours, and the reaction mixture was not homogenous because of the precipitation of product. Significant amount of the unidentified by-products were formed after heating at 90° C. for 4 hours.

### Example 7

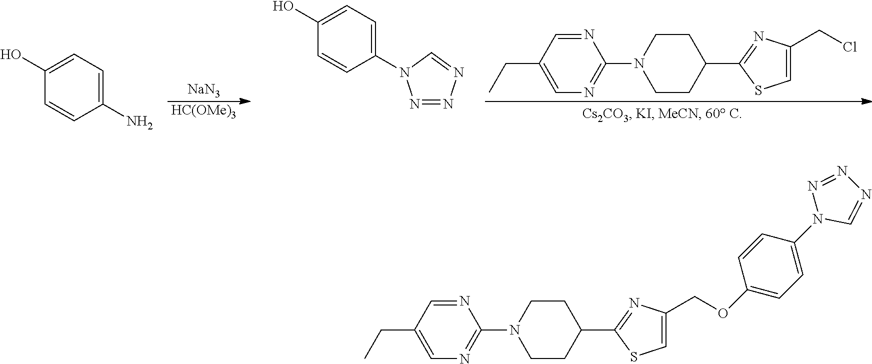

### 4-Tetrazol-1-yl-phenol

To a Kimax tube (25×150 mm) were added 4-aminophenol (200 mg, 1.83 mmol), sodium azide (167 mg, 2.57 mg, 1.4 eq.), acetic acid (1 mL), 2 drops of concentrated hydrochloride acid, and trimethyl orthoformate (0.5 mL) at room temperature. The mixture was stirred, and heated up to 100° C. on a heating block. After at 100° C. for 20 min, the temperature was lowered to 80° C., and water (1 mL) was added. When the mixture was cooled down to room temperature, the liquids were removed using pipette. The solid was washed with water (1 mL×3) and heptane (1 mL), and tried under vacuum. The white solid was used in the next step without further purification.

TLC: hexane-ethyl acetate 50:50, Rf (product)=0.28; Rf (starting material)=0.23, UV and iodine positive.

¹HNMR (400 MHz, D₃COD), δ 9.58 (s, 1H), 7.61 (d, J=9.0 Hz, 2H), 6.97 (d, J=9.0 Hz, 2H) ppm.

To the same tube from above reaction (with the synthesized 4-tetrazol-1-yl-phenol in) were added 2-[4-(4-Chloromethyl-thiazol-2-yl)-piperidin-1-yl]-5-ethyl-pyrimidine (571-110, 532 mg, 1.65 mmol), Cs₂CO₃ (596 mg, 1.83 mmol), KI (14 mg) in acetonitrile (2 mL). The mixture was heated at 60° C. for 10 hours (The reaction was followed by HPLC/MS).

After cooling the reaction mixture was treated with ethyl acetate (100 mL) and water (20 mL). The water phase was separated out. The organic phase was washed with brine (20 mL), dried over anhydrous sodium sulfate, concentrated. The residue was dissolved in small amount of dichloromethane and purified by 40 g silica gel Combiflash column to afford 580 mg (70% yield in two steps) of desired product as white solid.

¹H NMR (DMSO-d₆): δ 9.98 (1H, s), 8.24 (2H, s), 7.80 (2H, d, J=6.8 Hz), 7.66 (1H, s), 7.28 (2H, d, J=6.8 Hz), 5.20 (2H, s), 4.67 (2H, m), 3.32 (1H, m), 3.01 (2H, m), 2.43 (2H, q, J=7.2 Hz), 2.07 (2H, m), 1.59 (2H, m), 1.11 (3H, t, J=7.2 Hz) ppm. MS (ESI), m/z 449.

1. A method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof 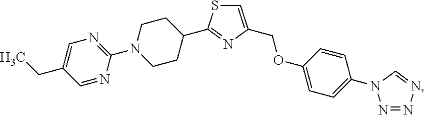
the method comprising contacting in dimethylformamide, in the presence of base, a compound of Formula (IX) with a compound of Formula (X) wherein L is a leaving group selected from the group consisting of F, Cl, Br, I, OS(O)₂CF₃, OS(O)₂CH₃ and OS(O)CF₃. 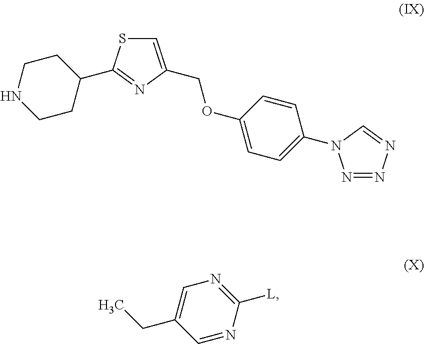
and optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

2. The method of claim 1 wherein the compound of Formula (IX) and (X) are contacted at a temperature of 60° C. to 120° C.

3. The method of claim 2 wherein the temperature is 70° C. to 90° C.

4. The method of claim 3 wherein the temperature is 79° C. to 81° C.

5. The method of claim 4 wherein the temperature is 80° C.

6. The method of claim 1 wherein the base is selected from the group consisting of NaOH, Na₂CO₃, NaHCO₃, KHCO₃, K₂CO₃, Cs₂CO₃, Et₃N, and i-Pr₂NEt.

7. The method of claim 1 wherein the compound of Formula (IX) is prepared by contacting a compound of Formula (VIII) with acid

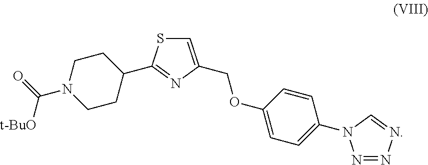

8. The method of claim 7 wherein the compound of Formula (VIII) is prepared by contacting a compound of Formula (VI) with a compound of Formula (VII)

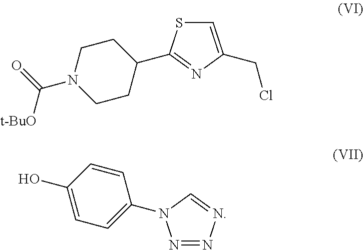

9. The method of claim 8 wherein the compounds of Formula (VI) and Formula (VII) are contacted in a polar organic solvent selected from dimethyl formamide (DMF) and acetonitrile (MeCN) and in presence of base.

10. The method of claim 9 wherein the solvent is MeCN.

11. The method of claim 9 wherein the solvent is DMF.

12. The method of claim 9 wherein the base is Et₃N.

13. The method of claim 9 wherein the base is Cs₂CO₃.

14. The method of claim 9 wherein the base is NaHCO₃.

15. The method of claim 8 wherein the compound of Formula (VI) is prepared by contacting a compound of Formula (IV) with a compound of Formula (V)

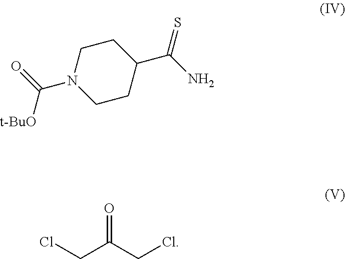

16. The method of claim 15 wherein the compounds of Formula (IV) and Formula (V) are refluxed in a polar organic solvent in presence of base.

17. The method of claim 16 wherein the base is selected from the group consisting of Na₂CO₃, K₂CO₃, Cs₂CO and MgCO₃.

18. The method of claim 8 wherein the compound of Formula (VII) is prepared by contacting 4-aminophenol with sodium azide and trimethylorthoformate.

19. The method of claim 15 wherein the compound of Formula (IV) is prepared by contacting a compound of Formula (II) with a compound of Formula (III)

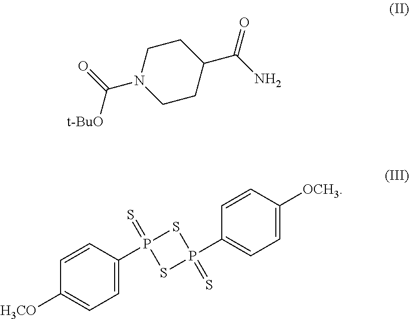

20. The method of claim 19 wherein the compound of Formula (II) is prepared by contacting a compound of Formula (I) with di-tert-butyl dicarbonate (Boc₂O).

21. A method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof 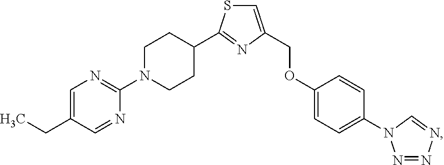
the method comprising:
(a) contacting a compound of Formula (I) with di-tert-butyl dicarbonate (Boc₂O) to form a compound of Formula (II) 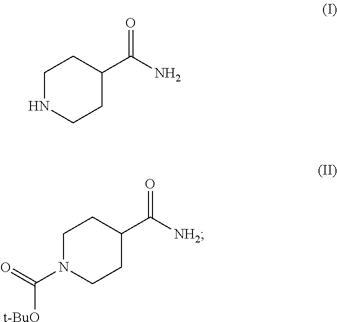
(b) contacting the compound of Formula (II) with a compound of Formula (III) to form a compound of Formula (IV) 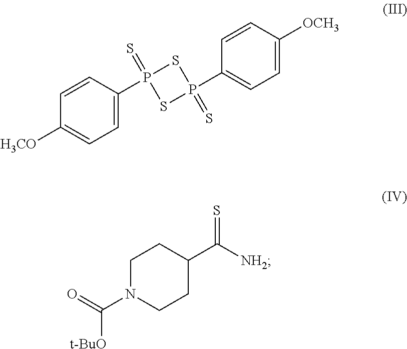
(c) contacting the compound of Formula (IV) with a compound of Formula (V) to form a compound of Formula (VI) 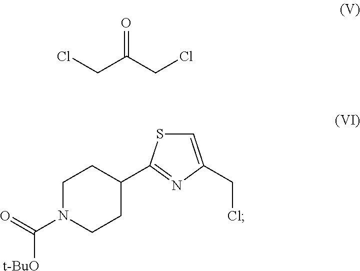
(d) contacting the compound of Formula (VI) with a compound of Formula (VII) to form a compound of Formula (VIII) 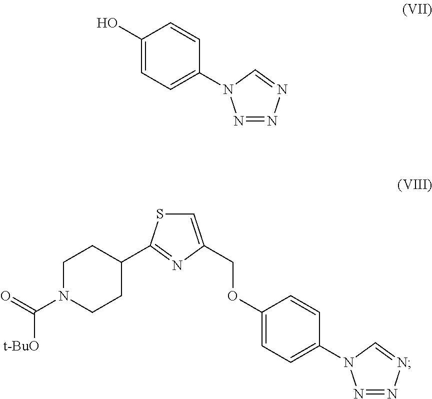
(e) contacting the compound of Formula (VIII) with acid to form a compound of Formula (IX) 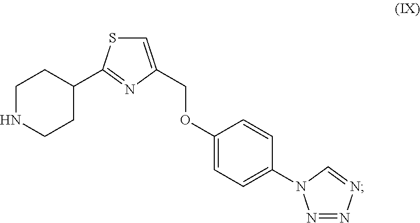
(f) contacting in dimethylformamide in presence of base the compound of Formula (IX) with a compound of Formula (X) wherein L is a leaving group selected from the group consisting of F, Cl, Br, I, OS(O)₂CF₃, OS(O)₂CH₃ and OS(O)CF₃, 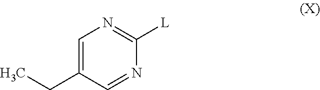
to form 5-ethyl-2-{4-[4-(4-tetrazol-1-yl-phenoxymethyl)-thiazol-2-yl]-piperidin-1-yl}-pyrimidine; and
(g) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

22. The method of claim 21 wherein the compound of Formula (IX) and (X) are contacted at a temperature of 60° C. to 100° C.

23. The method of claim 22 wherein the temperature is 70° C. to 90° C.

24. The method of claim 23 wherein the temperature is 79° C. to 81° C.

25. The method of claim 24 wherein the temperature is 80° C.

26. A method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof 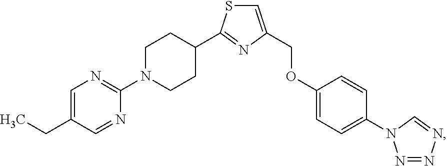
comprising contacting a compound of Formula (XXIV) with a compound of Formula (VII) in presence of base selected from the group consisting of NaOH, Na₂CO₃, K₂CO₃, Cs₂CO₃ and NaH 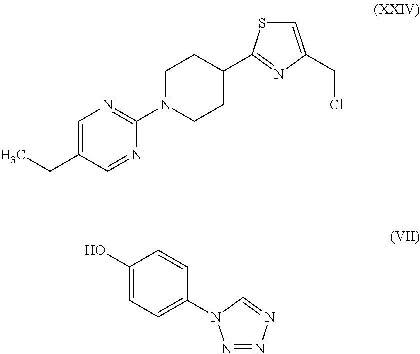
and optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

27. A method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof 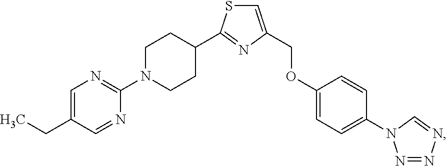
the method comprising:
(a) contacting a compound of Formula (I) with a compound of Formula (XXI) wherein T is a leaving group selected from the group consisting of F, Cl, Br, I, OS(O)₂CF₃, OS(O)₂CH₃ and OS(O)CF₃ to form a compound of Formula (XXII) 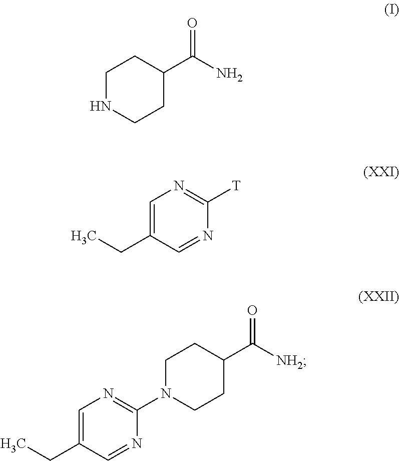
(b) contacting the compound of Formula (XXII) with a compound of Formula (III) to form a compound of Formula (XXIII) 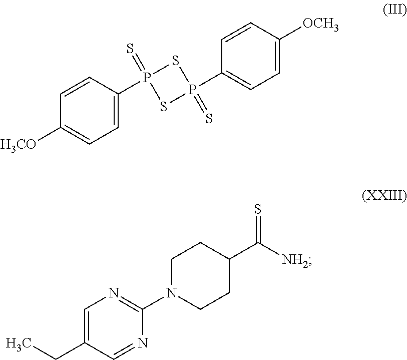
(c) contacting the compound of Formula (XXIII) with a compound of Formula (V) to form a compound of Formula (XXIV) 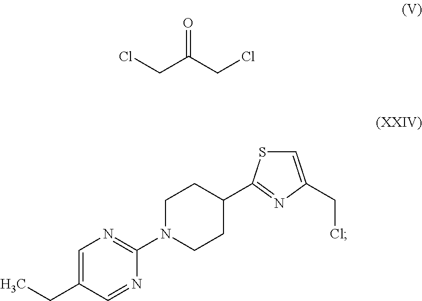
(d) contacting the compound of Formula (XXIV) with a compound of Formula (VII) 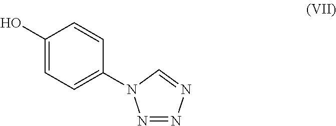
to form 5-ethyl-2-{4-[4-(4-tetrazol-1-yl-phenoxymethyl)-thiazol-2-yl]-piperidin-1-yl}-pyrimidine; and
(e) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

28. A method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof 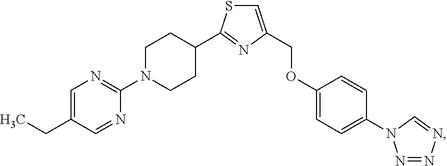
the method comprising:
(a) contacting a compound of Formula (IV) with acid to form a compound of Formula (XI) 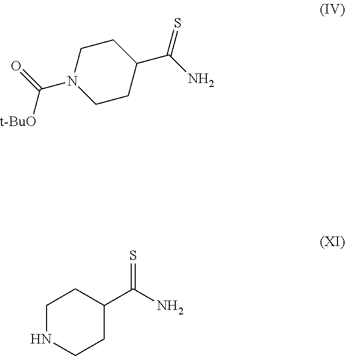
(b) contacting a compound of Formula (XXI) wherein T is a leaving group selected from the group consisting of F, Cl, Br, I, OS(O)₂CF₃, OS(O)₂CH₃ and OS(O)CF₃ to form a compound of Formula (XXIII) 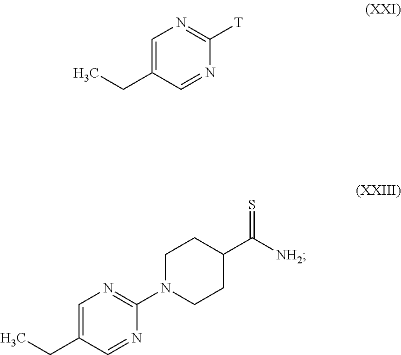
(c) contacting the compound of Formula (XXIII) with a compound of Formula (V) to form a compound of Formula (XXIV) 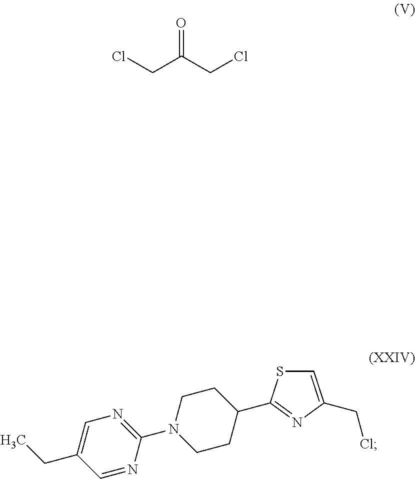
(d) contacting the compound of Formula (XXIV) with a compound of Formula (VII) 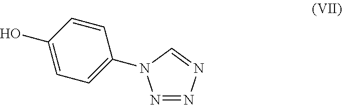
to form 5-ethyl-2-{4-[4-(4-tetrazol-1-yl-phenoxymethyl)-thiazol-2-yl]-piperidin-1-yl}-pyrimidine; and
(e) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

29. A method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof 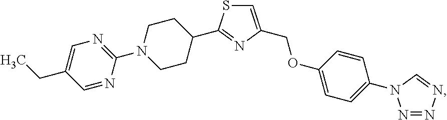
the method comprising:
(a) contacting the compound of Formula (XXIII) with a compound of Formula (XXIV) to form a compound of Formula (XXV) 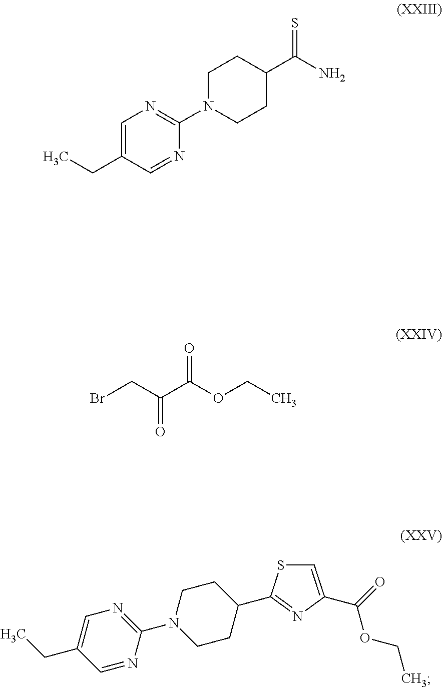
(b) contacting the compound of Formula (XXV) with a reducing agent to form a compound of Formula (XXVI) 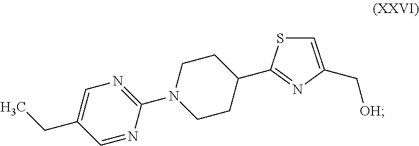
(c) contacting the compound of Formula (XXVI) with a compound of Formula (VII) 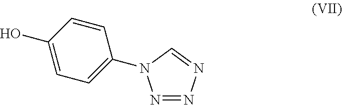
under Mitsunobu coupling conditions to form 5-ethyl-2-{4-[4-(4-tetrazol-1-yl-phenoxymethyl)-thiazol-2-yl]-piperidin-1-yl}-pyrimidine; and
(d) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

30. A method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof 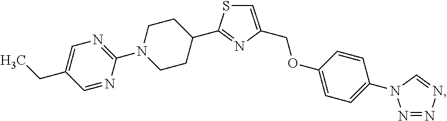
the method comprising:
(a) contacting the compound of Formula (XXIII) with a compound of Formula (XXIV) to form a compound of Formula (XXV) 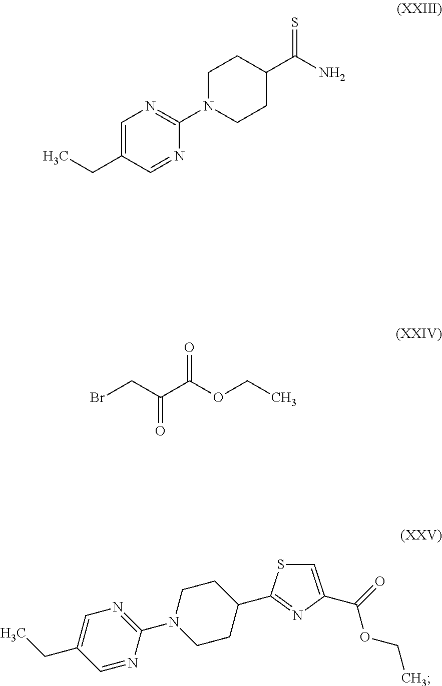
(b) contacting the compound of Formula (XXV) with a reducing agent to form a compound of Formula (XXVI) 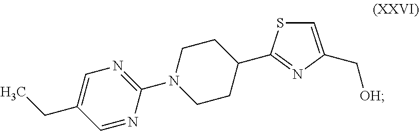
(c) converting the compound of Formula (XXVI) to a compound of Formula (XXVII) wherein Q is a leaving group selected from the group consisting of Cl, Br, I, OS(O)₂CF₃, OS(O)₂CH₃ and OS(O)CF₃ 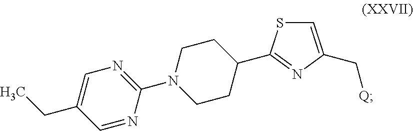
(d) contacting the compound of Formula (XXVII) with a compound of Formula (VII) 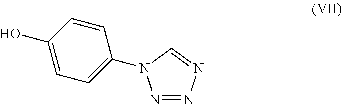
to form 5-ethyl-2-{4-[4-(4-tetrazol-1-yl-phenoxymethyl)-thiazol-2-yl]-piperidin-1-yl}-pyrimidine; and
(e) optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

31. A method for preparing 5-ethyl-2-{4-[4-(4-tetrazol-1-yl-phenoxymethyl)-thiazol-2-yl]-piperidin-1-yl}-pyrimidine or a pharmaceutically acceptable salt thereof 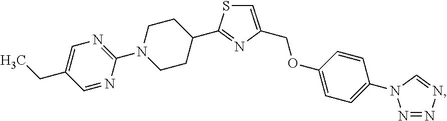
comprising contacting a compound of Formula (XXVII) wherein Q is a leaving group selected from the group consisting of Cl, Br, I, OS(O)₂CF₃, OS(O)₂CH₃ and OS(O)CF₃ with a compound of Formula (VII) in presence of base selected from the group consisting of NaOH, Na₂CO₃, K₂CO₃, Cs₂CO₃ and NaH 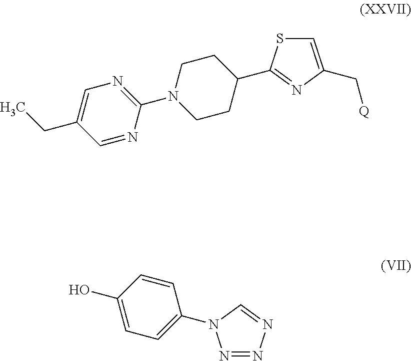
and optionally contacting 5-ethyl-2-{4-[4-(4-tetrazol-1-yl-phenoxymethyl)-thiazol-2-yl]-piperidin-1-yl}-pyrimidine with a pharmaceutically acceptable acid to form a pharmaceutically acceptable salt thereof.

32. The method of claim 1 for preparing the pharmaceutically acceptable salt of 5-ethyl-2-{4-[4-(4-tetrazol-1-yl-phenoxymethyl)-thiazol-2-yl]-piperidin-1-yl}-pyrimidine.

33. The method of claim 32 wherein the salt is a HCl salt.

34. An intermediate compound for use in the preparation of 5-ethyl-2-{4-[4-(4-tetrazol-1-yl-phenoxymethyl)-thiazol-2-yl]-piperidin-1-yl}-pyrimidine selected from the group consisting of 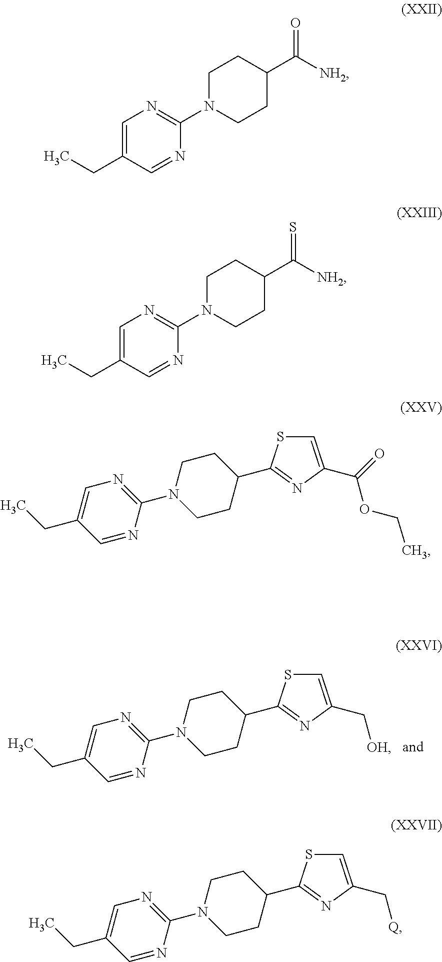
wherein Q is a leaving group selected from the group consisting of Cl, Br, I, OS(O)₂CF₃, OS(O)₂CH₃ and OS(O)CF₃.
